(19) United States
(12) Patent Application Publication
(10) Pub. No.: US 2021/0077079 A1
(43) Pub. Date: Mar. 18, 2021

(54) Reusable specimen imaging device holder system with replaceable membranes

(71) Applicants: Janice Ann Cureton, Salinas, CA (US); Matthew Lucas Chatman, Prunedale, CA (US)

(72) Inventors: Janice Ann Cureton, Salinas, CA (US); Matthew Lucas Chatman, Prunedale, CA (US)

(21) Appl. No.: 16/575,372

(22) Filed: Sep. 18, 2019

(51) Int. Cl. A61B 10/00; B01L 9/00; G01N 33/483

(52) U.S. Cl. CPC A61B 10/0096; B01L 9/52; G01N 2223/6126; B01L 2300/0609; B01L 2300/0809; G01N 33/4833

(57) ABSTRACT

The present invention is for the retention of surgically removed tissue for radiographic imaging. In breast conservation surgery specimen radiographic imaging is used to evaluate the success of removed diseased tissue with a negative margin. Accurate positioning of tissue orientation with minimal compression of the removed pathologic neoplasm is employed. The radiolucent device holder's purpose is to secure the tissue in proper orientation for radiographic imaging and margin evaluation. The present invention is a reusable system. It features a snap together component that allows for the replacing of the radiolucent membranes. Components are properly disinfected and membranes replaced making the device holder ready for reuse. This reusability is innovative, more cost effective and reduces medical waste. While this present invention, reusable specimen imaging holder device system with replaceable membranes has particular use in breast conservation surgery specimen imaging for optimal margin evaluation it is not exclusive to that purpose.

### BACKGROUND

### 1. Field of the Invention

This invention relates to an apparatus for holding surgically excised breast tissue for radiographic imaging for the purpose of optimal tissue margin evaluation.

### 2. Brief Description of the Related Art

My passion for this invention began after our hospital bought four new intraoperative radiographic units for imaging our breast specimens at the various hospital surgical sites. We began using a new specimen device holder called the Wedge by Faxitron, Bioptics, prior art referenced Akif, B., U.S. Pat. No. 8,741,232. Our breast radiologists prefer a true 90 degree orthogonal view for breast specimen imaging for margin evaluation. Although, the Wedge was very unique in its new design and methodology, our radiologist did not feel it resulted in a true 90 degree orthogonal view with its rotational component, but rather felt it resulted in more of a 45 degree view. We continue using the Wedge as it provides adequate retention to hold the specimen in proper orientation with minimal compression of the surgical specimen; however, we are dismantling the rotational component and not utilizing its full design and methodology. We also investigated other specimen holders; prior art referenced Devlin et al., U.S. Pat. No. 5,383,472, and Hansen et al., U.S. Pat. No. 8,162,140, both which did not provide the necessary tension to hold the specimen in place with proper orientation of the surgical markings. The process of creating a different image holder that fit our needs began to formulate. The end result of those ideas and models is the invention of the reusable specimen imaging device holder system with replaceable membranes. Its reusability is innovative, resulting in cost savings and reduced medical waste. Prior art referenced Garrigus, George W., U.S. Pat. No. 4,837,795, and Lary, B., U.S. Pat. No. 4,993,056 were two other device systems used in the past for this same purpose, and they were also non-disposable. However, both were criticized in prior art referenced Devlin et al., U.S. Pat. No. 5,383,472, due to their cleaning process and potential for cross contamination of tissue particles, and concern for excess compression of the surgical specimen. Unlike those non-disposable devices sited in the previously mentioned prior art, the reusable specimen image holder device system with replaceable membranes has a plastic framework that is easily sanitized with medical grade sanitizing products preventing blood and body fluid contamination to healthcare workers. The replaceable transparent membranes prevent any concern for cross contamination of tissue particles. It also features minimal compression of the surgical specimen with adequate retention to hold the excised tissue in proper positioned margin orientation for radiographing and evaluation by the medical team.

Early stage breast cancers are treated with breast conservation surgery and radiation therapy or mastectomy. Breast conservation surgery is the removal of the diseased tissue with negative surgical margins and acceptable postsurgical cosmetic results. A negative surgical margin is defined as no ink on the tumor. A team of medical professionals assist the surgeon in their evaluation of the tissue margins. Prior to breast conservation surgery the patient has usually had a procedure called a localization. It is usually done by a radiologist who places guide wires which “goal post” the diseased tissue. There are numerous methods for accomplishing localizations prior to the patient's surgery, wire, seeds, or one of the latest techniques occurs at biopsy where a radiofrequency clip is placed, the surgeon uses a radiofrequency monitoring unit to assist in localizing the clip and aids the surgeon in the removal of adequate diseased tissue.

The National Comprehensive Cancer Network has established guidelines for the medical team to aid in providing optimal margin evaluation. Optimal margin evaluation provides the surgeon and medical team with valuable information needed to hopefully prevent the need for another surgery, called a re-excision. Approximately one in five surgeries, or 20% must go for a second surgery due to positive margins. A positive margin becomes a prediction of local recurrence for the patient. The need for more surgeries can create emotional stress and financial hardship, as well as increased risk for a poorer cosmetic result, and could lead to mastectomy in some cases. These guidelines have three requirements. The first is orientation of the surgical specimen, there are various methods used to accomplish proper orientation, many different ones can be used. We use S.A.M. markings at our facilities. Second, there must be a description of the surgical gross and microscopic margin status. Third, it must include a reporting of the distance, orientation and type of tumor (invasive or ductal cancer in situ) in relation to the closest margin.

A specimen radiograph is useful when a surgeon is preforming breast conservation surgery to remove the diseased tissue and a surgical margin with no cancer cells. Current technology uses either the conventional radiographic method where the specimen would be transported and imaged in the radiology department, or the latest technology makes use of the intraoperative imaging units that are in the surgical suites or in close proximity to them. When the intraoperative units are used, the specimen is placed in a radiolucent specimen retention holder with proper positioning and orientation, and then radiographed. The radiographic image of the specimen is viewed by the radiologist and surgeon and provides correlation and evaluation of the excised tissue providing important feedback of the surgical margins. Surgical margin evaluation plays an important role in breast conservation surgery.

The present invention of a reusable radiolucent retention device holder is useful and necessary to hold the removed diseased tissue in place for imaging with proper orientation for optimal margin evaluation. The invention herein, relates to an apparatus for retaining a surgically excised tissue specimen for radiographic imaging for tissue margin evaluation. Its purpose is to hold the excised tissue specimen in proper radiographic orientation as positioned by the technologist or surgical staff. It is valuable to the surgeon, radiologist, and pathologist in their evaluation of adequate removal of diseased tissue margins. The invention is non-disposable, making it more cost effective, and environmentally friendly. The construction of the reusable system utilizes two transparent film membranes ranging from 0.005 to 0.1 inch thickness, held in tension to support the excised tissue for radiographic imaging purposes. It is composed of four components designed to snap together. All components are constructed from Pla+ or abs plastic. There are two main structure components which snap together using a ball and socket hinge to form a clamp in which the tissue to be examined is supported between the transparent membranes. There are two snap together components that secure the transparent membranes to the two main structural frames. All plastic components can be sanitized after each use with medical grade sanitizers to prevent any contamination of blood or body fluids to the healthcare team. The transparent/radiolucent membranes are replaced after each use thus preventing any cross contamination of tissue particles. While this present invention, reusable specimen image holder device system with replaceable membranes has particular use in breast conservation surgery, it is not exclusive to that purpose. See the following detailed summary of the embodiment.

### SUMMARY

The embodiments disclosed herein relate to a reusable system for retaining a surgically excised tissue sample for radiographic imaging. The construction of the reusable specimen imaging holder device utilizes two transparent film membranes ranging from 0.005 to 0.1-inch thickness, held in tension to support excised tissue for radiographic imaging purposes. CV 4 is composed of four components designed to snap together. All components are constructed from pla+ or abs plastic. CV 4.1 and CV 4.2 are the main structure components and snap together using a ball and socket hinge to form a clamp in which the tissue to be examined is supported between the transparent membranes. CV 4.3 is needed twice per assembly and is used to secure the transparent film membrane to the components 4.1 and 4.2 individually. CV 4.3 latches onto CV 4.1 and CV 4.2 using seven static clips that fit into surface recesses of CV 4.1 and CV 4.2. To install the membrane to either CV 4.1 or CV 4.2, the membrane can be laid over the face of either structural components and then secured and tensioned by latching CV 4.3 on over the top of the membrane. CV 4.4 is an “L” shaped bracket that becomes the static latch that will hold the hinge of CV 4.1 and CV 4.2 closed when the specimen is being radiographed. CV 4.4 has a tapered tab that snaps into a cavity of CV 4.1 that joins the two together and will remain attached during use. CV 4.2 has a recessed surface that CV 4.4 will clip into when the hinge is closed, preventing the hinge from opening while the tissue is in place and being radiographed.

Once both structural components (CV 4.1 and CV 4.2) have membranes secured to them and CV 4.4 is in place on CV 4.1, the tissue sample to be radiographed can be placed on the surface of the membrane on CV 4.1, and then the specimen will be “sandwiched” between each membrane when the hinge is closed and latched. CV 4.1 and CV 4.4 are the only two parts that connect and disconnect during normal use. To clean or change CV 4 unsnap CV 4.3 from CV 4.1 and CV 4.2, disinfect and replace the transparent film.

There is a limited availability of surgical specimen image holders that minimally compress the excised tissue and offer reusability. Our system provides a simplified and inexpensive alternative to current one time use device holders. It can provide reduced cost and reduced medical waste associated with these procedures. Because our invention features a plastic framed device that is easily sanitized with medical grade sanitizers, and allows for replacement of the radiolucent membrane that prevents cross contamination of tissue particles it is a useful tool in radiographic specimen imaging.

### BRIEF DESCRIPTION OF THE DRAWINGS

FIG. 1 shows a perspective view of CV4 and how the parts interact with each other.

FIG. 2 shows CV 4.1 from a perspective view and a bottom view.

FIG. 3 shows CV 4.2 in a perspective view and a bottom view.

FIG. 4 shows CV 4.3 in two perspective views, top and bottom and a bottom view.

FIG. 5 shows how the hinge works.

FIG. 6 shows how CV 4.4 interacts with CV 4.1.

### BRIEF SUMMARY

The invention is a reusable device holder used to retain surgically excised tissue for radiographic imaging. The reusable device holder has a framework with a bottom and top unit with a snap together component all made from pla+ or abs plastic. Two replaceable transparent film membranes are held in tension to support the excised tissue in proper specimen orientation for radiographic imaging purposes. The imaging process allows for radiologic, surgical and pathologic correlation of the excised tissue margins.

### DETAILED DESCRIPTION OF THE EMBODIMENTS

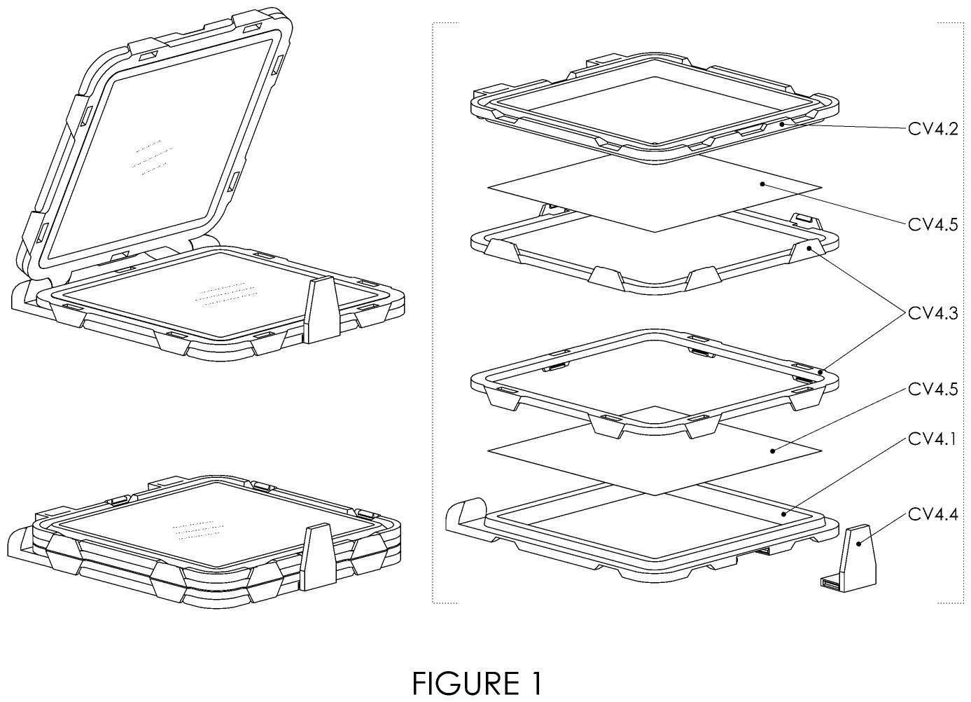

FIG. 1 shows a fully assembled perspective view of the embodiment. It is composed of CV 4.1, CV 4.2, two pieces of CV4.3, CV.4, and CV 4.5.

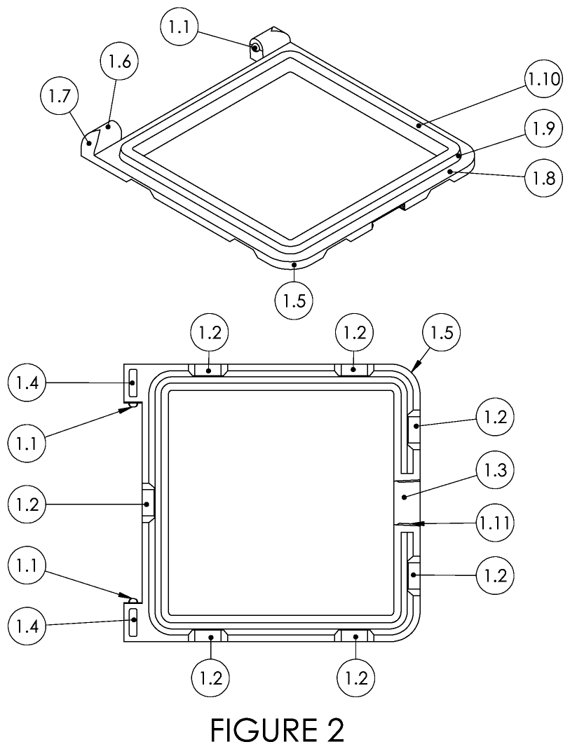

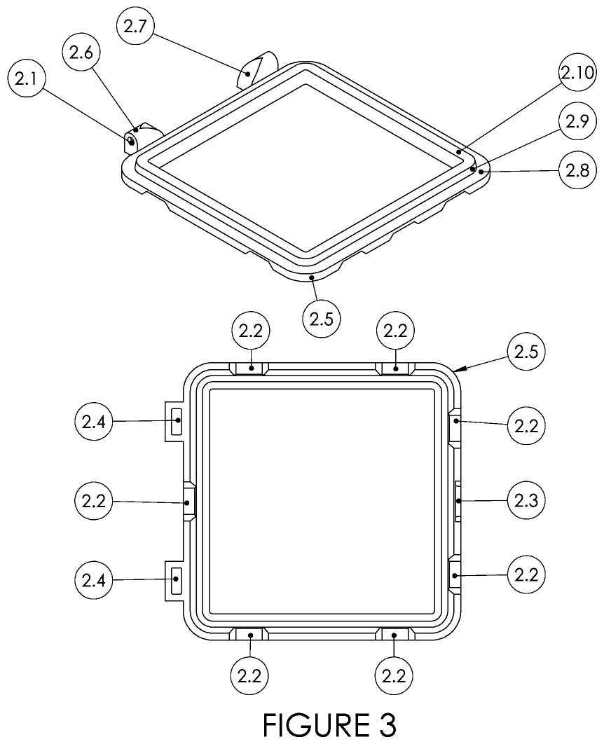

FIG. 2 shows a perspective view and a bottom view of CV4.1. It identifies the individual working components.
1.1 is a convex sphere that makes up half of the working hinge.
1.2 is the shelf that allows the clip on component 3.1.
1.3 is a cut out to allow room for CV 4.4 to be inserted.
1.4 is a cavity to lower thickness of material to optimize even shrinkage of the medium.
1.5 is a curved edge to aid in ease of use.
1.6 is the structure to maximize support for the hinge features.
1.7 is curved and reduces material.
1.8 is one of four surfaces that trap the working membrane on CV 4.1.
1.9 is the second of four surfaces that trap the working membrane on CV 4.1.
1.10 is one of two surfaces that push the two membranes together to form the outer limits where the specimen will rest.
1.11 consist of a protruding ledge that fits into the cut out of 4.2 to secure half of the main latch.
2. FIG. 3 is CV4.2 in a perspective and bottom view and identifies each working part.
2.1 is a concave sphere that is the second part of the hinge.
2.2 is the shelf that 3.1 clips into.
2.3 is a cut out that allows 4.1 to clip onto to form the main latch.
2.4 is a cavity to lower thickness of material to optimize even shrinkage of the medium.
2.5 is a curved edge which aids in ease of use.
2.6 is curved to allow maximum structure to support the hinge features.
2.7 further reduces material and aids in ease of use.
2.8 is the first of four surfaces used to trap the working membrane on CV 4.2.
2.9 is the second of the four surfaces used to trap the working membrane.
2.10 is the second surface used to push the two membranes together to form the outer limit of where the specimen will rest.

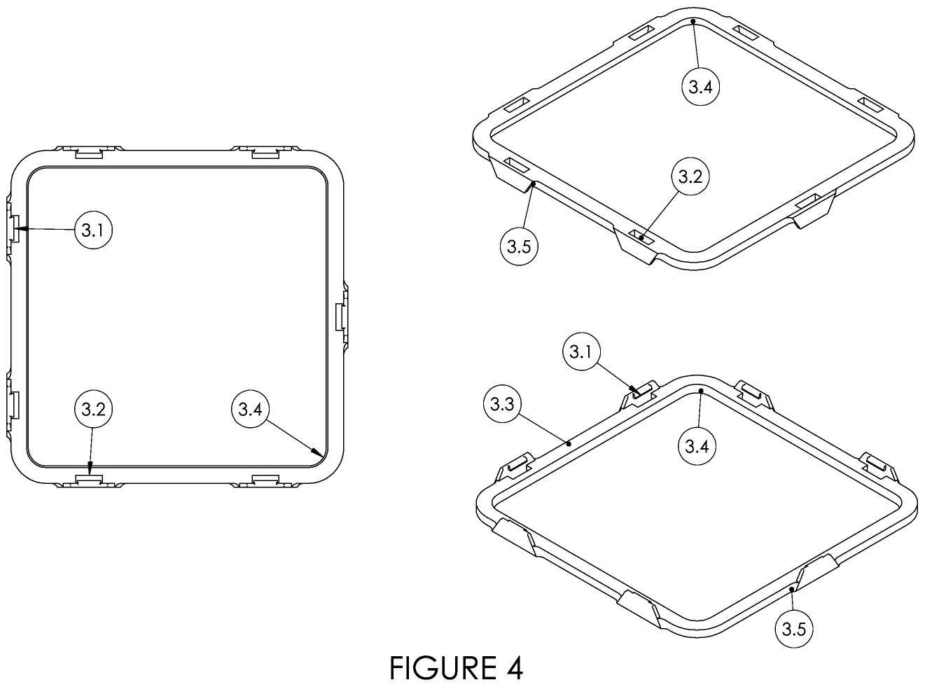

FIG. 4 shows CV4.3 in two perspective views, top and bottom, and bottom.
3.1 is the part that clips into the shelf piece 2.2. This latch mechanism allows for release and changing of the membrane.
3.2 is a cut out to allow ease of injection molding.
3.3 is one of two surfaces that trap the working membrane to either CV 4.1 or CV 4.2.
3.4 is the second textured face that is used to trap the working membrane to either CV4.1 or CV 4.2.
3.5 is the curved latch mechanism which allows for release and product durability and usage.

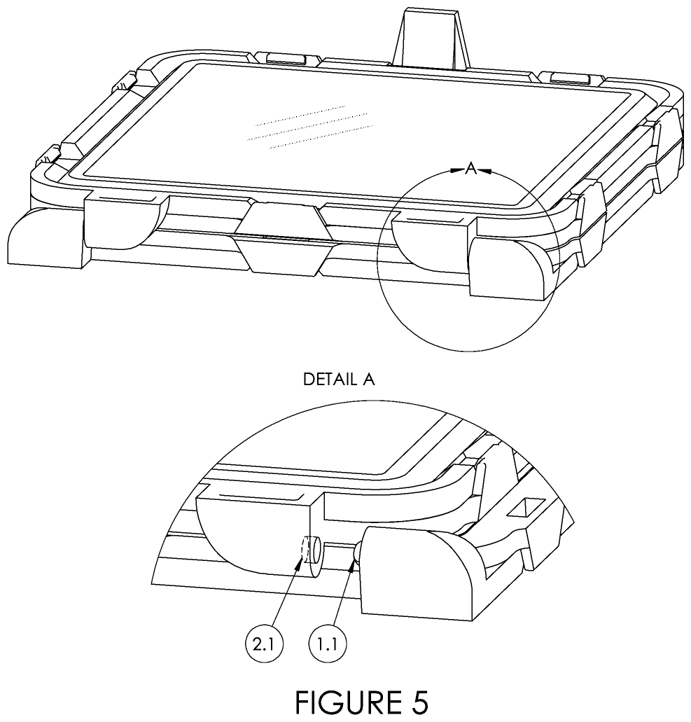

FIG. 5 is a detailed view of the hinge and how it works with the other perspective parts 1.1 and 2.1 from FIG. 1 and FIG. 2.

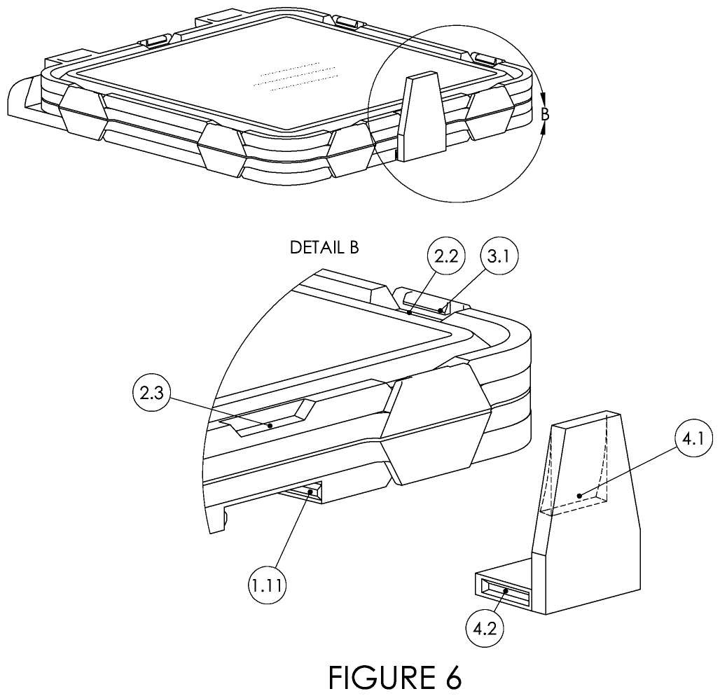

FIG. 6 is a detailed view showing the interactive parts 1.1, 2.2, 2.3, 3.1 and the latch component 4.1 and 4.2.

While the invention has been described in conjunction with specific embodiments thereof, it is evident that modifications and variations will be apparent to those skilled in the art. According, embodiments of the invention as set forth herein are intended to be illustrative, not limiting. There are changes that may be made without departing from the spirit and scope of the invention.

What is claimed is:

1. The method of a reusable specimen imaging device holder system which utilizes replaceable membranes. The device holder system may be provided in a plurality of sizes and shapes and still applicable to the system in the method claimed.

2. The method in claim 1 where the structural components of the device holder have a top and bottom unit, CV4.1 and CV 4.2 with a snap on component that secures the membranes to them, (CV 4.3).

3. The method in claim 1 of placing the tissue sample for radiographic imaging occurs by placing the sample on the surface of the membrane on CV 4.1, allowing the sample to be “sandwiched” between each membrane of the holder, then closing and latching the holder. (CV 4.4)

4. The method in claim 1 where the device may be disinfected and the membranes changed for reuse.
